SEQUENCE LISTING (8 sequences)

SEQ ID NO: 1 - protein, 117 aa, synthetic construct
MQGVQVVESGGGLMQAGGSLRLSCAASGFPVDSAHMHWVRQAPGKGLEWIGEIHHSGSTYYNPSLKSRVTISRDNSKNTLYLQMNNLRAEDTAIYYCAVDTFGADYWGQGTQVTVSS

SEQ ID NO: 2 - protein, 125 aa, synthetic construct
DVQLQASGGGSVQAGGSLRLSCTASESTFDLYVMGWFRQAPGKGPEWVSGINADGSNTHYTDSVKGRFTISRDNSKNTLYLQMNALKPEDTAVYYCAADPATPGSEKPAYHYDYWGQGTQVTVSS

SEQ ID NO: 3 - protein, 117 aa, synthetic construct
EVQLQASGGRLVQAGGSLRLSCAASGRTFSAGTMGWFRQAPGKQREAVSAIAGTDGIYYTDSVKGRFTISRYNAKNTAYLQMNNLRAEDTAIYYCNIGRVTPYADFWGQGTQVTVSS

SEQ ID NO: 4 - protein, 120 aa, synthetic construct
MAVQLVESGGGLVQAGGSLRLSCAASGYDFSSYWMHWVRQAPGKALEWVSGISDHGYTNYADSVKGRFTISRDNAKNTVYLQMNSLQPEDTGVYYCAARYGNTLAYYDYWGQGTQVTVSS

SEQ ID NO: 5 - protein, 120 aa, synthetic construct
MAIQLVESGGGLVQAGGSLRLSCAASGRTFSSLAMAWFREAPGKEREWVAAIVWTGDRTHYADFVKGRFTISRDNALNTVSLQMNNLKPVDTAVYYCAAGRSLLSDYAYWGQGTQVSVAS

SEQ ID NO: 6 - protein, 125 aa, synthetic construct
MAVQLVESGGGLVQAGGSLRLSCAASGFIFGRNAMGWYRQAPGKEREGVSCISSGGGRTSYADSVKGRFTISRDNAKNTVYLQMNNLRAEDTAIYYCAAAGYGQELSYHYDYWGQGTQVTVSSAR

SEQ ID NO: 7 - protein, 120 aa, synthetic construct
EVQLVESGGGLVQAGGSLRLSCVASRRLFEYYTMGWYRQAPGKQREFVAAISWSGGGSSYADSVKGRFTISRDNAKNTVYLQMNNLKPEDSAVYYCAADPALRYSDSWGQGTQVTVSSAR

SEQ ID NO: 8 - protein, 118 aa, synthetic construct
MAVQLVESGGGLVQAGGSLRLSCAASGRTFSAGTMGWFRQAPGKEREFVAAIRWSGGSAYYADSVKGRFTISRDNGKNTVYLQMNSLKLEDTAVYYCASSRPGTVNYWGKGTQVTVSS

(19) United States
(12) Patent Application Publication
(10) Pub. No.: US 2026/0337256 A1
(43) Pub. Date: Oct. 29, 2026

(54) USING ARTIFICIAL INTELLIGENCE (AI) TO DESIGN SINGLE DOMAIN ANTIBODIES THAT BIND VENEZUELAN EQUINE ENCEPHALITIS VIRUS

(71) Applicant: The Government of the United States of America, as represented by the Secretary of the Navy, Arlington, VA (US)

(72) Inventors: Scott N. Dean, Springfield, VA (US); Jinny L. Liu, Ellicott City, MD (US); Ellen R. Goldman, Germantown, MD (US)

(21) Appl. No.: 19/649,425

(22) Filed: Apr. 16, 2026

Related U.S. Application Data
(60) Provisional application No. 63/789,615, filed on Apr. 16, 2025.

(51) Int. Cl. C07K 16/116; G01N 33/569

(52) U.S. Cl. CPC C07K 16/116; G01N 33/56983; C07K 2317/565; C07K 2317/569; G01N 2333/181; G01N 2469/10

(57) ABSTRACT

Disclosed are design and characterization of Venezuelan equine encephalitis virus binding (VEEV-binding) single domain antibodies (sdAbs) produced with the assistance of a generative AI model-ProtGPT2-NRL. Utilizing a dataset of known alphavirus-binding sdAbs, ProtGPT2-NRL generated sequences with high predicted affinity and specificity for the E2 glycoprotein of VEEV. The multisequence alignment of the eight selected sdAbs were further evaluated and two of the eight were deemed to be most effective at binding.

### CROSS-REFERENCE TO RELATED APPLICATIONS

This patent application claims priority to U.S. Provisional Patent Application Ser. No. 63/789,615, filed Apr. 16, 2025; which is incorporated by reference herein in its entirety into this disclosure.

### FEDERALLY-SPONSORED RESEARCH AND DEVELOPMENT

The United States Government has ownership rights in this subject disclosure. Licensing inquiries may be directed to Office of Technology Transfer, US Naval Research Laboratory, 4555 Overlook Avenue SW, Code 1004, Washington, DC 20375, USA; +1.202.767.7230; techtran@nrl.navy.mil, referencing NC 212548.

### INCORPORATION BY REFERENCE

This patent application incorporates by reference the Sequence Listing XML file submitted herewith via the patent office electronic filing system having the file name “212548US2SEQ.xml” and created on Apr. 15, 2026 with a file size of 8,970 bytes.

### BACKGROUND OF THE SUBJECT DISCLOSURE

### Field of the Subject Disclosure

The present subject disclosure relates generally to single domain antibody design. More particularly, the present subject disclosure relates to using artificial intelligence (AI) to design single domain antibodies.

### Background of the Subject Disclosure

Venezuelan equine encephalitis virus (VEEV) poses a significant risk due to its potential for causing severe disease in humans and animals, its historical production as a biological weapon in several countries, and its transmission dynamics. It being highly infectious via aerosol inhalation makes it a potential biothreat, in addition to incidences of natural outbreaks where thousands of people have become infected. The attenuated TC-83 strain of VEEV, generated by 83 serial passages where it acquired 12 mutations, is widely utilized for research purposes after its development as a vaccine for laboratory workers and members of the US military. While the TC-83 vaccine is somewhat effective in preventing human disease, a large proportion of recipients do not seroconvert, and evidence in non-human primates suggests vaccination is not protective against aerosol exposure. Given the lack of an effective FDA-approved vaccine for VEEV, and absence of any approved vaccine or therapeutic for alphaviruses generally, various therapeutic avenues are being explored, with antibody therapeutics in particular being of considerable interest.

Monoclonal antibodies (mAbs) have been demonstrated to protect non-human primates against aerosolized VEEV, even 48 h after exposure. Similarly, neutralizing mAbs against the E2 glycoprotein have been demonstrated to protect mice against VEEV aerosol challenge. However, since mAbs are large (~150 kDa) and unstable as well as difficult and expensive to produce, they are not the best option for treatment, where administration in the field without cold storage may be necessary.

Single domain antibodies (sdAbs) are a promising alternative. Given their growing popularity with numerous examples to accumulate in databases of sequences and, critically, their size and relative ease in production, sdAbs have recently become a target for demonstration of protein design using generative deep learning models. These generative models have the ability to greatly speed up the discovery and design process that is normally used in identification of sdAb candidates.

A need exists for a more efficient and productive design technique of sdAbs for VEEV and other alpha viruses.

### SUMMARY OF THE SUBJECT DISCLOSURE

The present subject disclosure relates generally to designing single domain antibodies.

Single domain antibodies (sdAbs) are very small antibodies originally derived from heavy chain-only antibodies naturally produced by camelids and other organisms, such as cartilaginous fishes (sharks, etc.). Their discovery traditionally relies on inoculation of camelids with an antigen and subsequent screening experiments. Generative artificial intelligence (AI) models for text including text transformers (such as protein language models, PLMs) have begun to be applied to producing new sdAb sequences. This subject disclosure describes new sdAbs produced with the assistance of PLMs and subsequent structure prediction evaluation. These sdAbs bind and neutralize Venezuelan equine encephalitis virus (VEEV) in experiments, the first of their kind designed using generative AI.

The present subject disclosure presents, through the assistance of AI, a technical and practical solution to the field of sdAb research by streamlining the process of finding potential sdAb candidates for a given virus, in this case, VEEV. The use of AI is integrated into a practical application of sdAb research and discovery. Of course, the potential sdAb candidates are further examined to determine the most ideal candidates. The present subject disclosure is not fully AI created, but AI-assisted in discovering potential sdAb candidates. The candidates are further examined to determine suitability.

In one exemplary embodiment, the present subject disclosure is an isolated antibody. The isolated antibody includes a protein sequence comprising one or more single domain antibodies (sdAbs) selected from the group consisting of SEQ ID NOs. 1-8, wherein the antibody has binding activity against the Venezuelan equine encephalitis virus (VEEV).

In another exemplary embodiment, the present subject disclosure is a single domain antibody (sdAb). The single domain antibody includes three diverse complementarity determining regions (CDRs) identified as CDR1, CDR2, and CDR3, the antibody having an amino acid sequence with at least 80% identity to a sequence selected from the group consisting of SEQ ID NOs. 1-8 and an amino acid sequence identity in the CDR1, CDR2, and CDR3 regions of at least 75%; and wherein the antibody has binding activity against Venezuelan equine encephalitis virus (VEEV).

In yet another exemplary embodiment, the present subject disclosure is method of treating Venezuelan equine encephalitis virus (VEEV). The method includes obtaining a sample from a patient wherein the sample is known or suspected to contain VEEV; contacting the sample with a single domain antibody under conditions that permit antigen binding thereto; wherein at least a portion of any VEEV in the sample remains bound to the antibody, thereby producing a response indicative of the presence of VEEV in the sample; and if VEEV is detected in the sample, then providing the patient with suitable treatment against VEEV infection; wherein the single domain antibody comprises three diverse complementarity determining regions (CDRs) identified as CDR1, CDR2, and CDR3, the antibody having an amino acid sequence with at least 80% identity to a sequence selected from the group consisting of SEQ ID NOs. 1-8 and an amino acid sequence identity in the CDR1, CDR2, and CDR3 regions of at least 75%.

### BRIEF DESCRIPTION OF THE DRAWINGS

The patent or application file may contain at least one drawing executed in color. Copies of this patent or patent application publication with color drawing(s) will be provided by the Office upon request and payment of the necessary fee.

FIG. 1 depicts Clustal Omega alignment of sdAb sequences. CDRs 1-3 are highlighted. The characters “.”, “:”, and “*”, indicate degree of similarity of residues.

FIG. 2 shows a Table 1 of sdAb characteristics.

FIGS. 3A and 3B depict ELISAs of four sdAbs (V2B3 as positive control) with readings taken at 650 nm.

FIG. 3A shows A155, A16, A18, and A19 were evaluated for binding to VEEV TC-83 at a range of concentrations from 20 to 0.3125 μg/mL in uncoated or coated wells.

FIG. 3B shows area under the curve was calculated for each dose response. A155, A16, A18, and A19 (red) plotted against V2B3 (gray). Bars indicate the mean of ≥3 replicates and error bars indicate standard deviations.

FIG. 4 shows a Table 2 of PRNT50 value for TC83.

### DETAILED DESCRIPTION OF THE SUBJECT DISCLOSURE

The present subject disclosure addresses the shortcomings of VEEV antibody design, as discussed above.

### Definitions

Before describing the present subject disclosure in detail, it is to be understood that the terminology used in the specification is for the purpose of describing particular embodiments, and is not necessarily intended to be limiting. Although many methods, structures and materials similar, modified, or equivalent to those described herein can be used in the practice of the present subject disclosure without undue experimentation, the preferred methods, structures and materials are described herein. In describing and claiming the present subject disclosure, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of +10% of that stated.

### Purposes and Description of the Subject Disclosure

This subject disclosure presents the design and characterization of VEEV-binding sdAbs produced with the assistance of a generative AI model-ProtGPT2-NRL, which is Applicant's own version of the publicly available language model ProtGPT2. Utilizing a dataset of known alphavirus-binding sdAbs, ProtGPT2-NRL generated sequences with high predicted affinity and specificity for the E2 glycoprotein of VEEV. It should be noted that sdAbs found in this dataset are openly available, for example provided online: https://github.com/Jerome-Alvarez/TEMPRO. This is simply a collection from sequences already made available by others. The information in the dataset may be reproduced independently using public or open source datasets, code, and model weights

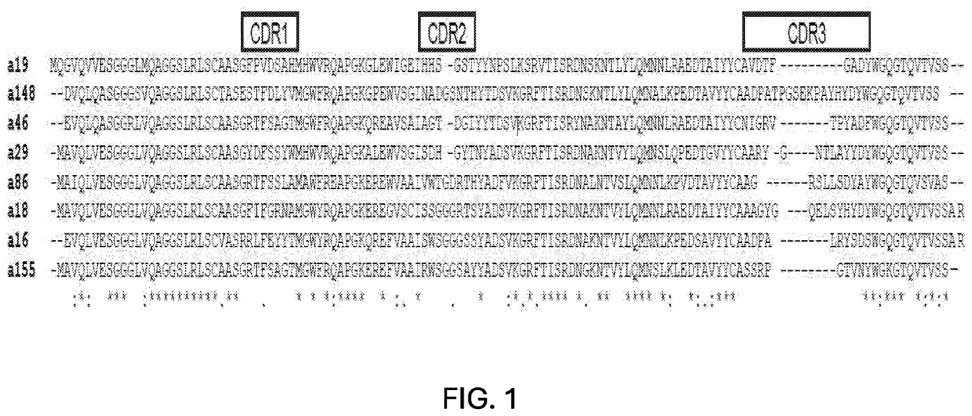

The multisequence alignment of the eight selected sdAbs is shown in FIG. 1. The specific amino acid sequences for the group of anti-VEEV sdAB are the subject of this disclosure. Anti-VEEV amino acid sequences are described as follows:

(a19)
SEQ ID NO: 1
MQGVQVVESGGGLMQAGGSLRLSCAASGFPVDSAHMHWVRQAPGKGL
EWIGEIHHSGSTYYNPSLKSRVTISRDNSKNTLYLQMNNLRAEDTAIYYC
AVDTFGADYWGQGTQVTVSS
(a148)
SEQ ID NO: 2
DVQLQASGGGSVQAGGSLRLSCTASESTFDLYVMGWFRQAPGKGPEWV
SGINADGSNTHYTDSVKGRFTISRDNSKNTLYLQMNALKPEDTAVYYCA
ADPATPGSEKPAYHYDYWGQGTQVTVSS
(a46)
SEQ ID NO: 3
EVQLQASGGRLVQAGGSLRLSCAASGRTFSAGTMGWFRQAPGKQREAV
SAIAGTDGIYYTDSVKGRFTISRYNAKNTAYLQMNNLRAEDTAIYYCNIG
RVTPYADFWGQGTQVTVSS
(a29)
SEQ ID NO: 4
MAVQLVESGGGLVQAGGSLRLSCAASGYDFSSYWMHWVRQAPGKALE
WVSGISDHGYTNYADSVKGRFTISRDNAKNTVYLQMNSLQPEDTGVYYC
AARYGNTLAYYDYWGQGTQVTVSS
(a86)
SEQ ID NO: 5
MAIQLVESGGGLVQAGGSLRLSCAASGRTFSSLAMAWFREAPGKEREWV
AAIVWTGDRTHYADFVKGRFTISRDNALNTVSLQMNNLKPVDTAVYYC
AAGRSLLSDYAYWGQGTQVSVAS
(a18)
SEQ ID NO: 6
MAVQLVESGGGLVQAGGSLRLSCAASGFIFGRNAMGWYRQAPGKEREG
VSCISSGGGRTSYADSVKGRFTISRDNAKNTVYLQMNNLRAEDTAIYYCA
AAGYGQELSYHYDYWGQGTQVTVSSAR
(a16)
SEQ ID NO: 7
EVQLVESGGGLVQAGGSLRLSCVASRRLFEYYTMGWYRQAPGKQREFV
AAISWSGGGSSYADSVKGRFTISRDNAKNTVYLQMNNLKPEDSAVYYCA
ADPALRYSDSWGQGTQVTVSSAR
(a155)
SEQ ID NO: 8
MAVQLVESGGGLVQAGGSLRLSCAASGRTFSAGTMGWFRQAPGKEREF
VAAIRWSGGSAYYADSVKGRFTISRDNGKNTVYLQMNSLKLEDTAVYY
CASSRPGTVNYWGKGTQVTVSS

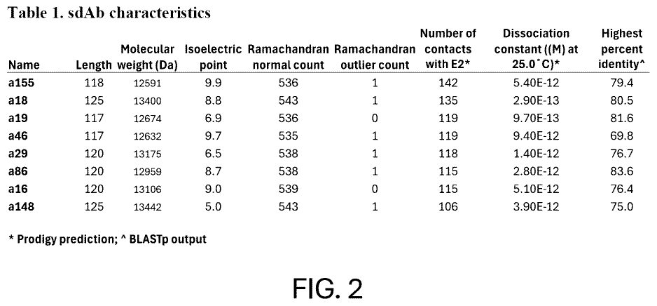

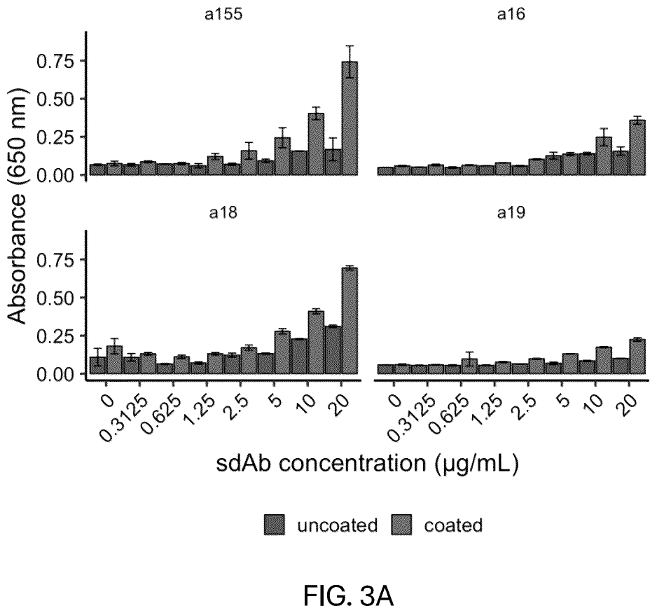

Characteristics of the generated sdAbs are shown in Table 1 as depicted in FIG. 2, including physicochemical features like isoelectric point, predicted features from AlphaFold-predicted structures like dissociation constant to E2 glycoprotein. Importantly, the sdAbs are not extremely similar to one another nor other known sdAbs, arrived at via all-vs-all BLASTp comparisons, where the highest identity is 83.6% for a86; this means each of the generated sequences is a non-obvious change from those sdAbs already present in the literature and/or in publicly available databases. These eight AI-assisted designed sdAbs were subsequently produced in Escherichia coli and purified for characterization. ELISA confirmed the binding efficacy of the sdAbs to irradiated TC-83 (FIGS. 3A-3B). Out of the eight sdAbs selected for production, two were of low solubility, and two were identified to not bind VEEV in preliminary ELISA experiments. From the four selected for evaluation, we identified that a18 and a155 were the best binders.

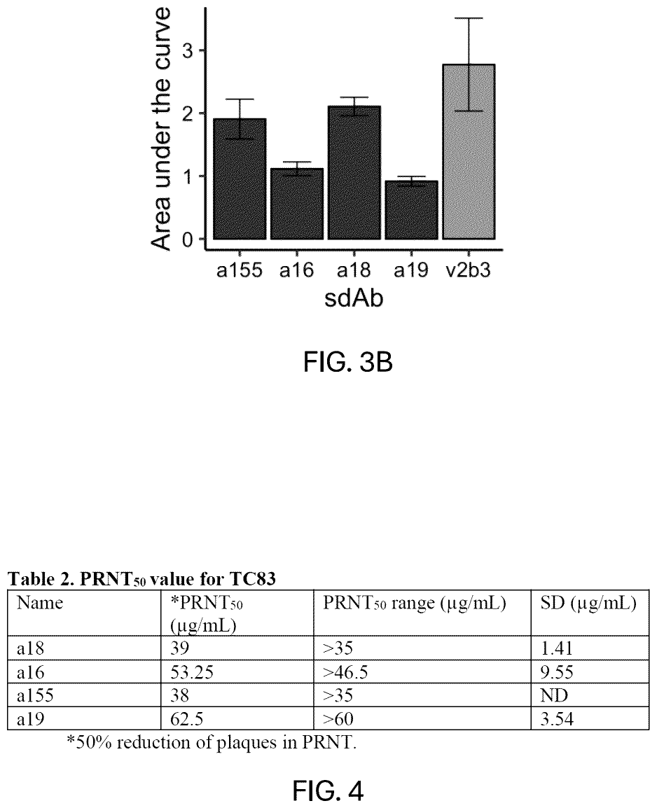

Using the four binders selected from ELISA experiments, plaque reduction neutralization tests (PRNTs) were performed. The PRNT assay showed that the sdAbs inhibited 50% VEEV infection in cell cultures at a range of ~35-65 μg/mL (PRNT50 values listed in Table 2 shown in FIG. 4). This research underscores the potential of generative AI in assisting in the development of antiviral therapeutics and provides a foundation for the rapid response to emerging viral threats and suggests an interesting and promising path for future antiviral drug discovery and development against VEEV and other alphaviruses.

### Advantages, New Features, and Alternatives

The present subject disclosure includes many advantages and new features, including, for example:
(1) The generative model used to generate these new sdAbs is capable of producing millions of unique sequences which can be computationally selected to have VEEV-binding characteristics.
(2) The sdAbs are distinct in sequence identity from known VEEV-binders.
(3) The sdAbs can serve as novel templates to build or improve upon, either by joining to other sdAbs in a bivalent approach or by modifying the sequences.

Alternatives to the subject disclosure described herein include several sdAbs previously identified by Liu et al.

A single domain antibody (sdAb) according to the present disclosure comprises three diverse complementarity determining regions (CDRs) identified as CDR1, CDR2, and CDR3, the antibody having an amino acid sequence with at least 80% identity to a sequence selected from the group consisting of SEQ ID NOs. 1-8 and an amino acid sequence identity in the CDR1, CDR2, and CDR3 regions of at least 75%; and wherein the antibody has binding activity against Venezuelan equine encephalitis virus (VEEV). Further, a medicament may be developed comprising a single domain antibody (sdAb) as recited herein, and a pharmaceutically-acceptable carrier. A medicament may be developed comprising a single domain antibody (sdAb) as recited herein, and a pharmaceutically-acceptable carrier. A nucleic acid may be developed encoding a single domain antibody a single domain antibody (sdAb) as recited herein. Further, the nucleic acid may encode single domain antibodies (sdABs) selected from the group consisting of SEQ ID NOs. 1-8, wherein the antibody has binding activity against the Venezuelan equine encephalitis virus (VEEV).

The following references, cited within this disclosure, are incorporated by reference herein in their entirety into this disclosure:
Liu, Jinny L., et al. “Bivalent single domain antibody constructs for effective neutralization of Venezuelan equine encephalitis.” Scientific Reports 12.1 (2022): 700.
Liu, Jinny L, et al., U.S. patent application Ser. No. 17/853,050, filed on Jun. 29, 2022, and entitled “Single Domain Antibodies That Bind and Neutralize Venezuelan Equine Encephalitis Virus.”

### CONCLUDING REMARKS

Although the present subject disclosure has been described in connection with preferred embodiments thereof, it will be appreciated by those skilled in the art that additions, deletions, modifications, and substitutions not specifically described may be made without departing from the spirit and scope of the subject disclosure. Terminology used herein should not be construed as being “means-plus-function” language unless the term “means” is expressly used in association therewith.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. An isolated antibody comprising a protein sequence comprising one or more single domain antibodies (sdAbs) selected from the group consisting of SEQ ID NOs. 1-8, wherein the antibody has binding activity against the Venezuelan equine encephalitis virus (VEEV).

2. The isolated antibody of claim 1, wherein the sdAb comprises SEQ ID NO. 6.

3. The isolated antibody of claim 1, wherein the sdAb comprises SEQ ID NO. 8.

4. A single domain antibody (sdAb) comprising three diverse complementarity determining regions (CDRs) identified as CDR1, CDR2, and CDR3, the antibody having an amino acid sequence with at least 80% identity to a sequence selected from the group consisting of SEQ ID NOs. 1-8 and an amino acid sequence identity in the CDR1, CDR2, and CDR3 regions of at least 75%; and
wherein the antibody has binding activity against Venezuelan equine encephalitis virus (VEEV).

5. A method of treating Venezuelan equine encephalitis virus (VEEV), comprising:
obtaining a sample from a patient wherein the sample is known or suspected to contain VEEV;
contacting the sample with a single domain antibody under conditions that permit antigen binding thereto; wherein at least a portion of any VEEV in the sample remains bound to the antibody, thereby producing a response indicative of the presence of VEEV in the sample; and
if VEEV is detected in the sample, then providing the patient with suitable treatment against VEEV infection;
wherein the single domain antibody comprises three diverse complementarity determining regions (CDRs) identified as CDR1, CDR2, and CDR3, the antibody having an amino acid sequence with at least 80% identity to a sequence selected from the group consisting of SEQ ID NOs. 1-8 and an amino acid sequence identity in the CDR1, CDR2, and CDR3 regions of at least 75%.

6. The method of claim 5, wherein the antibody is captured on a surface prior to said contacting.

7. The method of claim 5, wherein the antibody is used as a reporter.

8. The method of claim 5, wherein the patient is a non-human animal.
